(19) United States
(12) Patent Application Publication
(10) Pub. No.: US 2025/0198978 A1
(43) Pub. Date: Jun. 19, 2025

(54) SCREENING METHOD FOR RAW MATERIALS OF HIGH-YIELD SUSTAINABLE AVIATION FUEL

(71) Applicant: Hubei Tianji Bioenergy Co., Ltd., Xiaogan (CN)

(72) Inventors: Zhigang Tang, Xiaogan (CN); Minqiang Zhong, Xiaogan (CN); Tiancheng Deng, Xiaogan (CN); Hao Lu, Xiaogan (CN); Jinhua Deng, Xiaogan (CN); Xia Li, Xiaogan (CN); Chaohua Yi, Xiaogan (CN); Zhen Chen, Xiaogan (CN)

(21) Appl. No.: 18/957,659

(22) Filed: Nov. 23, 2024

(30) Foreign Application Priority Data
Dec. 13, 2023 (CN) 2023117122999

(51) Int. Cl. G01N 33/22

(52) U.S. Cl. CPC G01N 33/22

(57) ABSTRACT

Disclosed is a screening method for raw materials of high-yield sustainable aviation fuel, comprising the following steps: obtaining the fatty acid composition and content of different waste animal and vegetable oils; analyzing the positions of chemical bond cleavage during the hydrodeoxygenation, cracking, and isomerization processes of different fatty acids in waste animal and vegetable oils to produce sustainable aviation fuel, and obtaining the theoretical limit yields of sustainable aviation fuel prepared from different fatty acids; calculating the theoretical yields of sustainable aviation fuel produced from different waste animal and vegetable oils as raw materials based on their fatty acid composition and content; and selecting appropriate animal and vegetable oils as raw materials for producing sustainable aviation fuel based on the theoretical yields of sustainable aviation fuel. This disclosure provides guidance for enterprises to efficiently produce high-yield and high-value lipid-based bio-jet fuel.

### FIELD OF THE DISCLOSURE

The disclosure relates to the technical field of aviation fuel preparation, in particular to a screening method for raw materials of high-yield sustainable aviation fuel.

### BACKGROUND

Sustainable aviation fuel (SAF), also known as bio-jet fuel, is a new type of aviation fuel produced from raw materials such as waste animal and plant oils and fats (e.g., vegetable oils, animal and plant fats, waste cooking oils, etc.). Compared to traditional aviation fuels, SAF stands out for its cleanliness. Aviation fuels account for approximately 80% of the total carbon emissions in the aviation industry. Due to the need for long-distance flights with heavy loads and to cope with different flight environments, the aviation industry has stringent performance requirements for fuels and relies on fossil fuels, making it challenging to reduce carbon emissions. In the context of “carbon neutrality”, the reform and upgrading of aviation fuels have become an inevitable trend, and SAF is poised to encounter development opportunities.

In order to reduce greenhouse gas emissions and expand the raw material sources for aviation fuel, the development of bio-jet fuel production technologies has increasingly garnered attention from many countries, with oil-based bio-jet fuel process equipment technology being one of them. This technology converts plant oils or waste fats and oils into bio-jet fuel that can be used in aircraft through a series of complex chemical reactions and physical treatments. The emergence of this new technology not only addresses the issue of global surplus in edible oils but also solves the problem of food contamination caused by waste cooking oil and hogwash oil from the catering industry. At the same time, it opens up a new path for the sustainable development of the aviation industry with renewable bioenergy.

Currently, in the production of sustainable aviation fuel (SAF), particularly in the process of manufacturing new aviation fuels using waste animal and plant oils and fats (such as kitchen waste oil and hogwash oil) as raw materials, the yield rates among different companies vary significantly, ranging from 30% to 78%, with considerable variation.

### SUMMARY

The purpose of this disclosure is to provide a screening method for raw materials of high-yield sustainable aviation fuel to address the issues of low and unstable yield rates currently encountered in the production of SAF using waste animal and plant oils and fats.

In order to solve the above technical problems, this disclosure provides a screening method for raw materials of high-yield sustainable aviation fuel, comprising the following steps:
Step 1, obtaining composition and content of fatty acids in different waste animal and vegetable oils;
Step 2, analyzing the positions of chemical bond cleavage during the hydrodeoxygenation, cracking, and isomerization processes of different fatty acids in the waste animal and vegetable oils to produce sustainable aviation fuel, and obtaining the theoretical limit yield of sustainable aviation fuel prepared from different fatty acids;
when the fatty acids are unsaturated, the theoretical limit yield of sustainable aviation fuel prepared through the hydrodeoxygenation, cracking, and isomerization of unsaturated fatty acids is calculated based on the theoretical limit yield of alkanes produced from the hydrodeoxygenation of unsaturated fatty acids and the theoretical limit yield of sustainable aviation fuel generated from the cracking of these alkanes; the formula for calculating the theoretical limit yield of sustainable aviation fuel produced from unsaturated fatty acids is:

$${Q}_{i} = {B}_{i} \times {C}_{i}$$

where, Qᵢ represents the theoretical limit yield of sustainable aviation fuel produced from unsaturated fatty acids, Bᵢ represents the theoretical limit yield of alkanes produced from the hydrodeoxygenation of unsaturated fatty acids, and Cᵢ represents the theoretical limit yield of sustainable aviation fuel generated from the cracking of these alkanes after hydrodeoxygenation;

when the fatty acids are saturated, the theoretical limit yield of alkanes produced from the hydrodeoxygenation of saturated fatty acids is taken as the theoretical limit yield of sustainable aviation fuel produced from saturated fatty acids;

Step 3, based on the composition and content of fatty acids in waste animal and vegetable oils, as well as the theoretical limit yields of sustainable aviation fuel prepared from different fatty acids, calculating the theoretical yields of sustainable aviation fuel produced from different waste animal and vegetable oils as raw materials;

the theoretical yield calculation formula for sustainable aviation fuel is as follows:

$$η = {\sum }_{i = 1}^{n} ( {A}_{i} \times {P}_{i} )$$

where Aᵢ represents the content of different fatty acids in waste animal and vegetable oils, Pi represents the theoretical limit yield of sustainable aviation fuel prepared from different fatty acids, and η represents the theoretical yield of sustainable aviation fuel;

Step 4, Based on the theoretical yield of sustainable aviation fuel, selecting appropriate animal and vegetable oils as raw materials for producing sustainable aviation fuel;

in step 2, when analyzing the positions of chemical bond cleavage during the hydrodeoxygenation, cracking, and isomerization processes of different fatty acids in waste animal and vegetable oils to produce sustainable aviation fuel, the decarboxylation and decarbonylation reactions of fatty acids are not considered; additionally, the re-polymerization of carbon dioxide, ethane, propane, and naphtha produced during the production process, as well as the catalytic cracking reactions of long-chain alkanes, are also not taken into account.

Furthermore, the waste animal and vegetable oil is one or more of the following: waste cooking oil, hogwash oil, garbage oil, oleic acid-based vegetable oil, linoleic acid-based vegetable oil, and medium-chain fatty acid-based vegetable oil.

Furthermore, the oleic acid-based vegetable oil includes one or more of low-erucic acid rapeseed oil, high-erucic acid rapeseed oil, camellia oil, hogwash oil, palm oil, and olive oil; the linoleic acid-based vegetable oil include one or more of sunflower oil, cottonseed oil, and soybean oil; the medium-chain fatty acid-based vegetable oils include at least one of palm kernel oil and coconut oil.

Furthermore, when the animal and vegetable oil is primarily composed of unsaturated fatty acid glycerides, in step 3, the theoretical yield of sustainable aviation fuel produced using unsaturated fatty acid glycerides as the main raw material is calculated based on the composition and content of unsaturated fatty acids in the oils, as well as the theoretical limit yield of sustainable aviation fuel generated from the hydrodeoxygenation, cracking, and isomerization of unsaturated fatty acids.

Furthermore, when the animal and vegetable oil is primarily composed of saturated fatty acid glycerides, in step 3, the theoretical yield of sustainable aviation fuel produced using saturated fatty acid glycerides as the main raw material is calculated based on the composition and content of both saturated and unsaturated fatty acids in the oils, as well as the theoretical limit yield of sustainable aviation fuel.

Furthermore, the long-chain alkanes mentioned include pentadecane, heptadecane and octadecane.

Compared with existing technologies, the beneficial effects of this disclosure are: this disclosure, starting from the molecular structure of oils and fats, theoretically analyzes the potential positions of chemical bond cleavage during the hydrodeoxygenation, cracking, and isomerization processes of different fatty acids in oils and fats to produce alkanes (sustainable aviation fuel). It summarizes the logical relationship between the theoretical yield of sustainable aviation fuel and the fatty acid composition in oils and fats. Consequently, a screening method for raw materials of high-yield sustainable aviation fuel is provided, guiding the efficient and high-yield production of oil-based bio-jet fuel.

### DETAILED DESCRIPTION OF PREFERRED EMBODIMENTS

Below is a clear and comprehensive description of the technical solutions in the embodiments of this disclosure, combining with the embodiments. It is evident that the described embodiments are merely a portion of the embodiments of this disclosure, rather than exhaustive. Based on the embodiments presented in this disclosure, all other embodiments obtained by ordinary technicians in the field without creative effort fall within the scope of protection of this disclosure.

The screening method for raw materials of high-yield sustainable aviation fuel (bio-jet fuel) provided in the embodiments of this disclosure comprises the following steps:

(1) obtaining the fatty acid composition and content of different waste vegetable oils;

After consulting relevant literature review, it is known that the main unsaturated fatty acid composition of European low-erucic acid rapeseed oil is approximately 72% oleic acid, 12% linoleic acid, 5% linolenic acid, and 3% erucic acid. The total unsaturated fatty acid content is approximately 92%. The saturated fatty acid content is approximately 8%, among which palmitic acid accounts for about 6% and stearic acid accounts for about 2%.

The main unsaturated fatty acid composition of Chinese high-erucic acid rapeseed oil is approximately 13% oleic acid, 12% linoleic acid, 8% linolenic acid, 9% eicosenoic acid, and 51% erucic acid. The total unsaturated fatty acid content is approximately 93%. The saturated fatty acid content is approximately 7%, among which palmitic acid accounts for about 5% and stearic acid accounts for about 2%.

The main unsaturated fatty acid composition of hogwash oil is approximately 36% oleic acid, 29% linoleic acid, and 3% linolenic acid. The total unsaturated fatty acid content is around 71%. The saturated fatty acid content is approximately 29%, among which palmitic acid accounts for about 22% and stearic acid accounts for about 7%.

The major unsaturated fatty acid composition of soybean oil is as follows: approximately 28% oleic acid, about 53% linoleic acid, and roughly 5% linolenic acid. The total unsaturated fatty acid content is approximately 86%. The saturated fatty acid content is about 14%, among which palmitic acid accounts for approximately 10% and stearic acid accounts for about 4%.

The major unsaturated fatty acid composition of palm oil is as follows: approximately 40% oleic acid and about 10% linoleic acid. The total unsaturated fatty acid content is approximately 50%. The saturated fatty acid content is about 50%, among which palmitic acid accounts for approximately 45% and stearic acid accounts for about 5%.

The fatty acid composition of palm kernel oil is as follows: approximately 9% caprylic acid, about 10% capric acid, roughly 50% lauric acid, about 15% myristic acid, and approximately 5% palmitic acid. The unsaturated fatty acid oleic acid accounts for about 10%.

The fatty acid composition of coconut oil is as follows: approximately 8% caprylic acid, about 6% capric acid, roughly 49% lauric acid, about 17% myristic acid, and approximately 13% palmitic acid. The unsaturated fatty acid oleic acid accounts for about 6%, and the unsaturated fatty acid linoleic acid is about 1%.

(2) Analyzing the theoretical maximum yield of bio-jet fuel produced through hydrodeoxygenation, cracking, and isomerization of different fatty acids in oils and fats (without considering fatty acid decarboxylation and decarbonylation reactions, as well as the re-polymerization and utilization of carbon dioxide, ethane, propane, and naphtha generated during the production process. The catalytic cracking of long-chain alkanes such as pentadecane (C15 alkane), heptadecane (C17 alkane), and octadecane (C18 alkane) is also not taken into account.)

Biomass oils such as fatty acid glycerides, fatty acid esters, and fatty acids can be directly converted into linear hydrocarbon compounds through hydrodeoxygenation, resulting in biomass deoxygenated oil. After hydrodeoxygenation, the linear alkanes undergo hydrocracking and isomerization, yielding alkanes with a high degree of isomerization within the C8 to C16 range, which can be utilized as bio-jet kerosene.

The principle for preparing sustainable aviation fuel through hydrodeoxygenation, cracking, and isomerization of oils and fats is as follows:

First step: hydrodeoxygenation treatment: performing deoxygenation on the raw material fats and oils under a hydrogen atmosphere to convert unsaturated bonds to saturated bonds, thereby increasing the saturation and stability of the oils and fats. The result of hydrogenation and deoxygenation is the production of linear alkanes (normal alkanes). The reaction process is as follows:

$$Triglyceride + Fatty acid \frac{Catalyst}{} Deoxygenation \\ Unsaturated bond \frac{Hydrogenation}{} Normal alkane$$

Second step: cracking and isomerization: performing cracking and hydroisomerization of the linear alkanes under the action of a catalyst to obtain isoalkanes (bio-jet fuel).

The hydrogenation and deoxygenation of octanoic acid produces eight-carbon alkane octane. The theoretical maximum yield of bio-jet fuel from the extreme hydrodeoxygenation of octanoic acid and octanoic acid glycerides is 69%.

The hydrogenation and deoxygenation of decanoic acid produces ten-carbon alkane decane. The theoretical maximum yield of bio-jet fuel from the extreme hydrodeoxygenation of capric acid and capric acid glycerides is 74%.

The hydrogenation and deoxygenation of lauric acid produces twelve-carbon alkane. The theoretical limit yield of bio jet fuel obtained by extreme hydrodeoxygenation of dodecanoic acid and dodecanoic acid glycerides is 77%.

The hydrogenation and deoxygenation of myristic acid produces fourteen-carbon alkane. The theoretical maximum yield of bio-jet fuel from the extreme hydrodeoxygenation of tetradecanoic acid and tetradecanoate glycerides is 80%.

The hydrogenation and deoxygenation of palmitic acid produces sixteen-carbon alkane. The theoretical maximum yield of bio-jet fuel from the extreme hydrodeoxygenation of hexadecanoic acid and hexadecanoate glycerides is 82%.

Oleic acid (octadecenoic acid) undergoes deoxygenation, cracking, and isomerization, resulting in the complete cleavage of the double bond at the 9=10 position to produce two parts of nonane. The theoretical maximum yield of alkanes from the extreme hydrogenation and deoxygenation of oleic acid is 84%. The yield of bio-jet fuel from the catalytic cracking of the alkanes obtained through extreme hydrogenation and deoxygenation is 100%.

The deoxygenation, cracking, and isomerization of linoleic acid (octadecadienoic acid) results in the complete cleavage of the double bonds to produce one part of nonane, one part of hexane (solvent oil), and one part of propane (liquefied petroleum gas), with the double bonds located at positions 9=10 and 12=13. The theoretical maximum yield of alkanes from the extreme hydrodeoxygenation of linoleic acid is 84%. The yield of bio-jet fuel from the catalytic cracking of these alkanes, which have undergone extreme hydrodeoxygenation, is 50%, with 33.3% bio-solvent oil and 16.7% liquefied petroleum gas.

The deoxygenation, cracking, and isomerization of linolenic acid (octadecatrienoic acid) leads to the complete cleavage of the double bonds, resulting in the production of one part of nonane and three parts of propane (liquefied petroleum gas), with the double bonds located at positions 9=10, 12=13, and 15=16. The theoretical maximum yield of alkanes from the extreme hydrodeoxygenation of linolenic acid is 84%, the yield of bio jet fuel generated by catalytic cracking of alkanes with extreme hydrogenation and deoxygenation is 50%, and the yield of liquefied petroleum gas is also 50%.

The deoxygenation, cracking, and isomerization of eicosenoic acid leads to the complete cleavage of the double bond at position 11=12, resulting in the formation of one part of undecane and one part of nonane. The theoretical maximum yield of alkanes from the extreme hydrogenation and deoxygenation of eicosenoic acid is 85%. and the yield of bio jet fuel from extreme hydrogenation and deoxygenation of the alkanes through catalytic cracking is 100%.

The deoxygenation, cracking, and isomerization of erucic acid (docosenoic acid) leads to the complete cleavage of the double bond at position 13-14, resulting in the formation of one part of tridecane and one part of nonane. The theoretical maximum yield of alkanes from the extreme hydrodeoxygenation of erucic acid is 86%, and the yield of bio jet fuel from extreme hydrogenation and deoxygenation of the alkanes through catalytic cracking is 100%.

(3) Calculating the theoretical yield of sustainable aviation fuel prepared from different vegetable oils as raw materials based on the fatty acid composition of these oils and the theoretical maximum yield of sustainable aviation fuel obtained through the processes of extreme hydrogenation saturation and double bond removal, catalytic hydrogenation and deoxygenation, and subsequent catalytic cracking of different fatty acids.

3.1 Yield of bio-jet fuel using hogwash oil as raw material

The complete cleavage of the double bond in oleic acid (octadecenoic acid) results in a yield of nonane of 36%×84%×100%=30.24%, contributing to a bio-jet fuel yield of 30.24%.

The complete cleavage of the double bonds in linoleic acid (octadecadienoic acid) results in a yield of nonane of 29%×84%×50%=12.18%, contributing to a bio-jet fuel yield of 12.18%.

The complete cleavage of the double bonds in linolenic acid (octadecatrienoic acid) results in a yield of nonane of 3%×84%×50%=1.26%, contributing to a bio-jet fuel yield of 1.26%.

The theoretical yield of bio-jet fuel from hogwash oil is approximately 30.24%+12.18%+1.26%=43.68%. This means that the yield of bio-jet fuel produced from hogwash oil as a raw material is around 43%.

3.2 Yield of bio-jet fuel using low-erucic acid rapeseed oil as raw material

The complete cleavage of the double bond in oleic acid (octadecenoic acid) results in a yield of nonane of 72%×84%×100%=60.48%, contributing to a bio-jet fuel yield of 60.48%.

The complete cleavage of the double bonds in linoleic acid (octadecadienoic acid) results in a yield of nonane of 12%×84%×50%=5.04%, contributing to a bio-jet fuel yield of 5.04%.

The complete cleavage of the double bonds in linolenic acid (octadecatrienoic acid) results in a yield of nonane of 5%×84%×50%=2.10%, contributing to a bio-jet fuel yield of 2.10%.

The complete cleavage of the double bond in erucic acid (docosenoic acid) results in a yield of tridecane and nonane of 3%×86%×100%=2.58%, contributing to a bio-jet fuel yield of 2.58%.

The theoretical yield of bio-jet fuel from rapeseed oil (with low erucic acid content) is 60.48%+5.04%+2.10%+2.58%=70.20%. This means that the yield of bio-jet fuel produced from low-erucic acid rapeseed oil as a raw material is around 70%.

3.3 Yield of bio-jet fuel using high-erucic acid rapeseed oil as raw material

The complete cleavage of the double bond in oleic acid (octadecenoic acid) results in a yield of nonane of 13%×84%×100%=10.92%.

The complete cleavage of the double bonds in linoleic acid (octadecadienoic acid) results in a yield of nonane of 12%×84%×50%=0.84%.

The complete cleavage of the double bonds in linolenic acid (octadecatrienoic acid) results in a yield of nonane of 8%×84%×50%=3.36%.

The complete cleavage of the double bond in eicosenoic acid results in a yield of undecane and nonane of 9%×85%×100%=7.65%.

The complete cleavage of the double bond in erucic acid (docosenoic acid) results in a yield of tridecane and nonane of 51%×86%×100%=43.86%.

The theoretical yield of bio-jet fuel from rapeseed oil (with high erucic acid content) is 10.92%+5.04%+3.36%+7.65%+43.86%=70.83%. This means that the yield of bio-jet fuel produced from high-erucic acid rapeseed oil as a raw material is around 70%.

3.4 Yield of bio-jet fuel using soybean oil as raw material:

The complete cleavage of the double bond in oleic acid (octadecenoic acid) results in a yield of nonane of 28%×84%×100%=23.52%.

The complete cleavage of the double bonds in linoleic acid (octadecadienoic acid) results in a yield of nonane of 53%×84%×50%=22.26%.

The complete cleavage of the double bonds in linolenic acid (octadecatrienoic acid) results in a yield of nonane of 5%×84%×50%=2.10%.

The theoretical yield of bio-jet fuel from soybean oil is 23.52%+22.26%+2.10%=47.88%. This means that the yield of bio-jet fuel produced from soybean oil as a raw material is approximately 47%.

3.5 Yield of bio-jet fuel using palm oil as raw material:

The complete cleavage of the double bond in oleic acid (octadecenoic acid) results in a yield of nonane of 40%×84%×100%=33.6%.

The complete cleavage of the double bonds in linoleic acid (octadecadienoic acid) results in a yield of nonane of 10%×84%×50%=4.20%.

The theoretical yield of bio-jet fuel from palm oil is 33.6%+4.20%=37.80%. This means that the yield of bio-jet fuel produced from palm oil as a raw material is approximately 37%.

3.6 Yield of alkanes from hydrodeoxygenation, cracking, and isomerization of medium-chain fatty acid glycerides derived from palm kernel oil and coconut oil

Palm Kernel Oil:

Octane yield: 9%×69%=6.21%;

Decane yield: 10%×74%=7.4%;

Dodecane yield: 50%×77%=38.5%;

Tetradecane yield: 15%×80%=12%;

Hexadecane yield: 5%×82%=4.1%;

Nonane yield: 10%×84%=8.4%;

The total yield of bio-jet fuel from palm kernel oil is approximately 76.61%.

Coconut Oil:

Octane yield: 8%×69%=5.52%;

Decane yield: 6%×74%=4.44%;

Dodecane yield: 49%×77%=37.73%;

Tetradecane yield: 17%×80%=13.6%;

Hexadecane yield: 13%×82%=10.66%;

Nonane yield (first fraction): 6%×84%=5.04%;

Nonane yield (second fraction, considering a smaller proportion due to isomerization or side reactions): 1%×84%×50%=0.42%;

The total yield of bio-jet fuel from coconut oil is approximately 77.41%.

The yields of bio-jet fuel prepared from different oil sources are shown in Table 1 below.

TABLE 1

| | | | | | | Mainly Saturated | |
|---|---|---|---|---|---|---|---|
| | Mainly Unsaturated Fatty Acids | | | | | Fatty Acids | |
| | | Low-erucic | High-erucic | | | Palm | |
| Raw | Hogwash | acid | acid | Soybean | Palm | kernel | Coconut |
| Materials | oil | rapeseed oil | rapeseed oil | oil | oil | oil | oil |
| bio-jet | 43% | 70% | 70% | 47% | 37% | 76% | 77% |
| fuel yield | | | | | | | |

From the table above, it can be observed that among the raw materials mainly composed of unsaturated fatty acids, low-erucic acid rapeseed oil and high-erucic acid rapeseed oil have the highest theoretical yield for producing bio-jet fuel, approximately 70%. This indicates that using rapeseed oil to produce bio-jet fuel is scientifically sound, suitable, offers the highest yield, and generates the least amount of ineffective by-products. Therefore, to produce bio-jet fuel with a high yield, enterprises can prioritize rapeseed oil as the raw material. For the production of bio-jet fuel from medium-chain fatty acid glycerides mainly composed of saturated fatty acids, palm kernel oil and coconut oil have comparable theoretical yields. Enterprises can select the appropriate raw material based on actual circumstances.

In summary, this disclosure recognizes that current practitioners tend to approach problems from the perspective of hydrocarbon cracking in the petrochemical industry, whereas oils and fats are substances with entirely different chemical structures and distinct hydrogenation and cracking pathways compared to petroleum. This disclosure theoretically analyzes the logical relationship between the theoretical yield of sustainable aviation fuel and the fatty acid composition of oils and fats. By knowing the fatty acid composition of oils and fats, one can deduce the theoretical yield of sustainable aviation fuel produced from them. This provides guidance for enterprises to efficiently and cost-effectively produce high-yield, high-value oil-based bio-jet fuel.

It should be noted that all the embodiments mentioned above belong to the same inventive concept, and each embodiment has its own focus in description. For any aspects not fully described in a particular embodiment, reference can be made to the descriptions in other embodiments.

The embodiments described above merely represent the implementation methods of this disclosure, and the descriptions are relatively specific and detailed. However, this should not be construed as limiting the scope of this disclosure. It should be pointed out that for those skilled in the art, without departing from the concept of this disclosure, several modifications and improvements can still be made, which all fall within the protection scope of this disclosure. Therefore, the protection scope of this disclosure patent should be determined by the appended claims.

1. A screening method for raw materials of sustainable aviation fuel, comprising the following steps:
step 1, obtaining composition and content of fatty acids in different waste animal and vegetable oils;
step 2, analyzing positions of chemical bond cleavage during hydrodeoxygenation, cracking, and isomerization processes of different fatty acids in the waste animal and vegetable oils to produce sustainable aviation fuel, and obtaining theoretical limit yields of sustainable aviation fuel prepared from different fatty acids;
when the fatty acids are unsaturated, a theoretical limit yield of sustainable aviation fuel prepared through the hydrodeoxygenation, cracking, and isomerization of unsaturated fatty acids is calculated based on a theoretical limit yield of alkanes produced from the hydrodeoxygenation of unsaturated fatty acids and a theoretical limit yield of sustainable aviation fuel generated from the cracking of the alkanes; a formula for calculating the theoretical limit yield of sustainable aviation fuel produced from unsaturated fatty acids is:
$${Q}_{i} = {B}_{i} \times {C}_{i}$$
where, Qᵢ represents the theoretical limit yield of sustainable aviation fuel produced from unsaturated fatty acids, Bᵢ represents the theoretical limit yield of alkanes produced from the hydrodeoxygenation of unsaturated fatty acids, and Cᵢ represents the theoretical limit yield of sustainable aviation fuel generated from the cracking of the alkanes after hydrodeoxygenation;
when the fatty acids are saturated, a theoretical limit yield of alkanes produced from the hydrodeoxygenation of saturated fatty acids is taken as a theoretical limit yield of sustainable aviation fuel produced from saturated fatty acids;
step 3, based on the composition and content of fatty acids in waste animal and vegetable oils, as well as the theoretical limit yields of sustainable aviation fuel prepared from different fatty acids, calculating theoretical yields of sustainable aviation fuel produced from different waste animal and vegetable oils as raw materials;
wherein a theoretical yield calculation formula for sustainable aviation fuel is as follows:
$$η = {\sum }_{i = 1}^{n} ( {A}_{i} \times {P}_{i} )$$
where Aᵢ represents the content of different fatty acids in waste animal and vegetable oils, Pi represents the theoretical limit yields of sustainable aviation fuel prepared from different fatty acids, and n represents the theoretical yield of sustainable aviation fuel;
step 4, based on the theoretical yield of sustainable aviation fuel, selecting appropriate waste animal and vegetable oils as raw materials for producing sustainable aviation fuel;
wherein in step 2, when analyzing the positions of chemical bond cleavage during the hydrodeoxygenation, cracking, and isomerization processes of different fatty acids in waste animal and vegetable oils to produce sustainable aviation fuel, decarboxylation and decarbonylation reactions of fatty acids are not considered; and re-polymerization of carbon dioxide, ethane, propane, and naphtha produced during the production process, as well as catalytic cracking reactions of long-chain alkanes, are also not considered;
wherein the screening method for raw materials of sustainable aviation fuel further comprises:
performing deoxygenation on selected raw materials under a hydrogen atmosphere to convert unsaturated bonds to saturated bonds, to thereby obtain linear alkanes; and
performing the cracking and the hydroisomerization on the linear alkanes under an action of a catalyst to obtain the sustainable aviation fuel.

2. The screening method for raw materials of sustainable aviation fuel according to claim 1, wherein in step 1, the waste animal and vegetable oil is one or more selected from the group consisting of waste cooking oil, hogwash oil, garbage oil, oleic acid-based vegetable oil, linoleic acid-based vegetable oil, and medium-chain fatty acid-based vegetable oil.

3. The screening method for raw materials of sustainable aviation fuel according to claim 2, wherein the oleic acid-based vegetable oil comprises one or more selected from the group consisting of low-erucic acid rapeseed oil, high-erucic acid rapeseed oil, camellia oil, hogwash oil, palm oil, and olive oil; the linoleic acid-based vegetable oil comprises one or more selected from the group consisting of sunflower oil, cottonseed oil, and soybean oil; and the medium-chain fatty acid-based vegetable oil comprises at least one of palm kernel oil and coconut oil.

4. The screening method for raw materials of sustainable aviation fuel according to claim 1, when the waste animal and vegetable oil is primarily composed of unsaturated fatty acid glycerides, in step 3, calculating a theoretical yield of sustainable aviation fuel produced using unsaturated fatty acid glycerides as main raw material is calculated based on the composition and content of unsaturated fatty acids in the oils, as well as the theoretical limit yield of sustainable aviation fuel generated from the hydrodeoxygenation, cracking, and isomerization of unsaturated fatty acids.

5. The screening method for raw materials of sustainable aviation fuel according to claim 1, when the waste animal and vegetable oil is primarily composed of saturated fatty acid glycerides, in step 3, calculating a theoretical yield of sustainable aviation fuel produced using saturated fatty acid glycerides as main raw material based on the composition and content of both saturated and unsaturated fatty acids in the oils, as well as the theoretical limit yield of sustainable aviation fuel.

6. The screening method for raw materials of sustainable aviation fuel according to claim 1, wherein the long-chain alkanes comprises pentadecane, heptadecane and octadecane.
